(19) United States
(12) Patent Application Publication
(10) Pub. No.: US 2024/0288450 A1
(43) Pub. Date: Aug. 29, 2024

(54) SYSTEMS AND METHODS FOR MONITORING AND TREATING STROKE

(71) Applicant: Arizona Board of Regents on Behalf of the Univerity of Arizona, Tucson, AZ (US)

(72) Inventor: Kristian DOYLE, Tucson, AZ (US)

(21) Appl. No.: 18/570,834

(22) PCT Filed: Jun. 14, 2022
(86) PCT No.: PCT/US2022/033347

§ 371 Date: Dec. 15, 2023

Related U.S. Application Data
(60) Provisional application No. 63/211,065, filed on Jun. 16, 2021.

(51) Int. Cl. G01N 33/68; A61K 45/06

(52) U.S. Cl. CPC G01N 33/6896; A61K 45/06; G01N 2333/96491; G01N 2800/52

(57) ABSTRACT

The present disclosure relates to methods of determining and administering a treatment course of action. In particular, the present disclosure relates to compositions and methods for monitoring and treating stroke (e.g., with anti-inflammation therapy).

### CROSS-REFERENCE TO RELATED APPLICATION

The present application claims priority to U.S. Provisional Patent Application No. 63/211,065, filed Jun. 16, 2021, which is hereby incorporated by reference in its entirety.

### STATEMENT REGARDING FEDERALLY SPONSORED RESEARCH OR DEVELOPMENT

This invention was made with government support under Grant No. R01 NS096091, awarded by National Institutes of Health. The government has certain rights in the invention.

### FIELD OF THE INVENTION

The present disclosure relates to methods of determining and administering a treatment course of action. In particular, the present disclosure relates to compositions and methods for monitoring and treating stroke (e.g., with anti-inflammation therapy).

### BACKGROUND OF THE INVENTION

According to Centers for Disease Control and Prevention, more than 795,000 people in the United States have a stroke every year. About 610,000 of these are first or new strokes. Furthermore, more than 20% of stroke survivors will have neurodegeneration that will lead to the develop of dementia within one year of their stroke.

The most significant improvements occur in the first few weeks post-stroke, often reaching a relative plateau after 3 months with less significant recovery subsequently, especially concerning motor symptoms. Thus, it is crucial for patients to have an efficient and quick access to a proper diagnostic post-stroke for assessing and monitoring damage and recovery from stroke.

### SUMMARY OF THE INVENTION

There is a chronic inflammatory response to stroke that occurs in the infarcted area of the brain and causes further neurodegeneration in the weeks and months after stroke. Data described herein indicated that this inflammatory response is driven by overwhelmed lipid processing in immune cells processing the lipid rich brain tissue destroyed by the stroke. There are no peripheral biomarkers of this inflammatory response that could be used as a tool to identify patients in which this inflammatory response is occurring or used as a metric for assessing the effectiveness of drugs being used to treat the chronic inflammatory response to stroke. To address this need, experiments described herein discovered that levels of MMP12 in the blood correlate with chronic inflammation and neurodegeneration after stroke.

Accordingly, provided herein is a method of identifying chronic inflammation after stroke, comprising: a) assaying a sample from a subject that has had a stroke for the level of matrix metalloproteinase-12 (MMP12); and b) identifying said subject as having chronic inflammation when the level of MMP12 is increased relative to the level in a subject that has not had a stroke.

Further embodiments provide a method of treating chronic inflammation in a subject that has had a stroke, comprising: a) assaying a sample from a subject that has had a stroke for the level of MMP12; and b) administering an anti-inflammation therapy when the level of MMP12 is increased relative to the level in a subject that has not had a stroke.

Yet other embodiments provide a method of treating chronic inflammation in a subject that has had a stroke, comprising: a) assaying a sample from a subject that has had a stroke for the level of MMP12; b) administering an anti-inflammation therapy when the level of MMP12 is increased relative to the level in a subject that has not had a stroke; and c) repeating the assaying step after the administering step.

Still further embodiments provide a method of monitoring a treatment in a subject that has had a stroke, comprising: a) assaying a sample from a subject that has had a stroke for the level of MMP12; b) administering an anti-inflammation therapy when the level of MMP12 is increased relative to the level in a subject that has not had a stroke; and c) repeating the assaying step after the administering step.

Other embodiments provide the use of an anti-inflammation therapy to treat inflammation after stroke when the level of MMP12 in a sample from a subject is increased relative to the level in a subject that has not had a stroke.

The present invention is not limited to a particular sample type. Examples include but are not limited to, blood, a blood product (e.g., plasma), cells, or tissue.

In some embodiments, the stroke is an ischemic stroke. In some embodiments, the level of MMP12 is the level of MMP12 nucleic acid (e.g., mRNA) or polypeptide. In some embodiments, the level of MMP13 is also measured. In some embodiments, the anti-inflammation therapy reduces or prevents neurodegeneration in the subject.

The present invention is not limited to particular anti-inflammatory therapy. For example, in some embodiments, the therapy is an agent that blocks the activity of a cytokine (e.g., IL-1α, IL-1β, and IL-1Ra, or TNF-α). The agent is, for example, a small molecule, a nucleic acid, or an antibody. Examples of anti-inflammation therapies include but are not limited to cyclodextrin, 2-hydroxypropyl-β-cyclodextrin (HPβCD), AdipoRon, biographene quantum dots, anti-CD20 antibodies (e.g. Rituximab), anti-TREM2 antibodies, anti-CD36 antibodies, metformin, rapamycin, inhibitors of cyclic nucleotide phosphodiesterase (PDE) (e.g. Ibudilast), dimethyl fumarate, TLR antagonists and modulators, dimethyl itaconate, and neurotrophin receptor modulators (e.g. LM11A-31).

The present invention is not limited to particular intervals for assaying the levels of MMP12. Examples include one or more of 24 hours, 1 week, 2 weeks, 4 weeks, 7 weeks, and 6 months (e.g., 1 day to one week; one day to 2 weeks, one day to 4 weeks, one day to 7 weeks, one day to 6 months, 1 week to 2 weeks, 1 week to 4 weeks, 1 week to 7 weeks, one week to 6 months, 2 weeks to 4 weeks, 2 weeks to 7 weeks, 2 weeks to 6 months, 4 weeks to 7 weeks, 4 weeks to 6 months, or 7 weeks to 6 months, although other intervals are specifically contemplated) after stroke. In some embodiments, the assaying is repeated at an interval of, for example, daily, weekly, monthly, yearly, or another interval.

Additional embodiments provide the step of further comprising assaying the level of a marker of neurodegeneration (e.g., neurofilament light (NfL)).

Additional embodiments are described herein.

### DESCRIPTION OF THE FIGURES

FIG. 1. Neurofilament light (NfL), an established biomarker of neurodegeneration, is detectible in plasma for at least 7 weeks after stroke. N=3-11 per timepoint/group.

FIG. 2. Biomarkers of the chronic inflammatory response to stroke based on bulk RNA sequencing of the infarct at 7 weeks after stroke.

FIG. 3. Biomarkers of acute (24 hr) stroke. Multiplex immunoassay analysis of mouse plasma at 24 hours, 1 week, 2 weeks, 4 weeks, 7 weeks, and 6 months after stroke. N=5-14 per timepoint/group.

FIG. 4. Biomarkers of chronic stroke. Multiplex immunoassay analysis of mouse plasma at 24 hours, 1 week, 2 weeks, 4 weeks, 7 weeks, and 6 months after stroke. N=5-14 per timepoint/group.

FIG. 5. Negative data: Multiple plasma proteins were not detectable as biomarkers of stroke at any timepoint. Multiplex immunoassay analysis of mouse plasma at 24 hours, 1 week, 2 weeks, 4 weeks, 7 weeks, and 6 months after stroke. N=5-14 per timepoint/group.

### DEFINITIONS

To facilitate an understanding of the present invention, a number of terms and phrases are defined below:

As used herein, the terms “detect”, “detecting” or “detection” may describe either the general act of discovering or discerning or the specific observation of a detectably labeled composition.

As used herein, the term “subject” refers to any organisms that are screened using the diagnostic methods described herein. Such organisms preferably include, but are not limited to, mammals (e.g., humans).

The term “diagnosed,” as used herein, refers to the recognition of a disease by its signs and symptoms, or genetic analysis, pathological analysis, histological analysis, and the like.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragments are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues (e.g., biopsy samples), cells, and gases. Biological samples include blood products, such as plasma, serum and the like. Such examples are not however to be construed as limiting the sample types applicable to the present invention.

### DETAILED DESCRIPTION OF THE INVENTION

Chronic inflammation after stroke contributes to secondary neurodegeneration (Doyle et al., J Neuro, 2015) and correlates with overwhelmed lipid processing in immune cells (Chung et al., eNeuro, 2018). In addition, helping immune cells process lipid debris after stroke reduces secondary neurodegeneration (//www.biorxiv.org/content/10.1101/2021.05.03.442388v1). However, for successful translation of these findings it would be helpful to have a biomarker of damaging chronic inflammation after stroke, so that individuals treated with interventions can be monitored for drug efficacy using their plasma. Experiments described herein identified MMP12 as a marker for chronic inflammation after stroke.

Accordingly, provided herein is a method of identifying chronic inflammation after stroke, comprising: a) assaying a sample from a subject that has had a stroke for the level of matrix metalloproteinase-12 (MMP12); and b) identifying said subject as having chronic inflammation when the level of MMP12 is increased relative to the level in a subject that has not had a stroke.

In some embodiments, an anti-inflammation therapy is administered to the subject when the level of MMP12 is increased relative to the level in a subject that has not had a stroke. In some embodiments, levels of MMP12 are monitored after stroke and/or during anti-inflammation therapy (e.g., at regular intervals of daily, weekly, monthly, yearly, or other interval) to monitor inflammation in the subject. The levels of MMP12 can be used to determine a treatment course of action (e.g., starting, stopping, or changing anti-inflammation therapy).

In some embodiments, the stroke is an ischemic stroke. In some embodiments, the level of MMP12 is the level of MMP12 nucleic acid (e.g., mRNA) or polypeptide. In some embodiments, the anti-inflammation therapy reduces or prevents neurodegeneration in the subject.

In some embodiments, the level of MMP13 is also measured. MMP12 and MMP13 are part of a cluster of MMP genes on chromosome 11 at site 11q22.3 and their transcription is coordinated.

The present invention is not limited to particular anti-inflammatory therapy. For example, in some embodiments, the therapy is an agent that blocks the activity of a cytokine (e.g., IL-1α, IL-1β, and IL-1Ra, or TNF-α). The agent is, for example, a small molecule, a nucleic acid, or an antibody. Examples of anti-inflammation therapies used after stroke include but are not limited to 2-cyclodextrin, hydroxypropyl-β-cyclodextrin (HPβCD), AdipoRon, biographene quantum dots, anti-CD20 antibodies (e.g. Rituximab), anti-TREM2 antibodies, anti-CD36 antibodies, metformin, rapamycin, inhibitors of cyclic nucleotide phosphodiesterase (PDE) (e.g. Ibudilast), dimethyl fumarate, TLR antagonists and modulators, dimethyl itaconate, and neurotrophin receptor modulators (e.g. LM11A-31).

Additional embodiments provide the step of further comprising assaying the level of a marker of neurodegeneration (e.g., neurofilament light (NfL)).

The present disclosure is not limited to particular values for a reference level of MMP12 or related markers. In some embodiments, the reference level is the level of MMP12 in a subject that has not had a stroke. In some embodiments, the reference level is an average of a given population of cells or samples obtained from a representative number of patients. In some embodiments, the reference level is pre-set. In some embodiments, the reference level is based on the level of expression of MMP12 or in the patient (e.g., the level at the beginning of treatment).

The present disclosure is not limited to particular methods of measuring the level of expression of MMP12 or related genes. Any patient sample may be tested according to methods of embodiments of the present invention. By way of non-limiting examples, the sample may be tissue (e.g., a brain biopsy sample), blood, or a fraction thereof (e.g., plasma, serum, cells), or circulating tumor cells.

In some embodiments, the level of expression of MMP12 nucleic acid is determined using a variety of nucleic acid techniques, including but not limited to: nucleic acid sequencing; nucleic acid hybridization; and, nucleic acid amplification.

A variety of nucleic acid sequencing methods are contemplated for use in the methods of the presfcent disclosure including, for example, chain terminator (Sanger) sequencing, dye terminator sequencing, and high-throughput sequencing methods. Many of these sequencing methods are well known in the art. See, e.g., Sanger et al., Proc. Natl. Acad. Sci. USA 74:5463-5467 (1997); Maxam et al., Proc. Natl. Acad. Sci. USA 74:560-564 (1977); Drmanac, et al., Nat. Biotechnol. 16:54-58 (1998); Kato, Int. J. Clin. Exp. Med. 2:193-202 (2009); Ronaghi et al., Anal. Biochem. 242:84-89 (1996); Margulies et al., Nature 437:376-380 (2005); Ruparel et al., Proc. Natl. Acad. Sci. USA 102:5932-5937 (2005), and Harris et al., Science 320:106-109 (2008); Levene et al., Science 299:682-686 (2003); Korlach et al., Proc. Natl. Acad. Sci. USA 105:1176-1181 (2008); Branton et al., Nat. Biotechnol. 26(10):1146-53 (2008); Eid et al., Science 323:133-138 (2009); each of which is herein incorporated by reference in its entirety.

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., Life Technologies/Ion Torrent, and Pacific Biosciences, respectively.

Illustrative non-limiting examples of nucleic acid hybridization techniques include, but are not limited to, in situ hybridization (ISH), microarray, and Southern or Northern blot. In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

In some embodiments, MMP12 levels are detected using fluorescence in situ hybridization (FISH). In some embodiments, FISH assays utilize bacterial artificial chromosomes (BACs). These have been used extensively in the human genome sequencing project (see Nature 409: 953-958 (2001)) and clones containing specific BACs are available through distributors that can be located through many sources, e.g., NCBI. Each BAC clone from the human genome has been given a reference name that unambiguously identifies it. These names can be used to find a corresponding GenBank sequence and to order copies of the clone from a distributor.

The present invention further provides a method of performing a FISH assay on human cells (e.g., brain cells). Specific protocols are well known in the art and can be readily adapted for the present invention. Guidance regarding methodology may be obtained from many references including: In situ Hybridization: Medical Applications (eds. G. R. Coulton and J. de Belleroche), Kluwer Academic Publishers, Boston (1992); In situ Hybridization: In Neurobiology; Advances in Methodology (eds. J. H. Eberwine, K. L. Valentino, and J. D. Barchas), Oxford University Press Inc., England (1994); In situ Hybridization: A Practical Approach (ed. D. G. Wilkinson), Oxford University Press Inc., England (1992)); Kuo, et al., Am. J. Hum. Genet. 49:112-119 (1991); Klinger, et al., Am. J. Hum. Genet. 51:55-65 (1992); and Ward, et al., Am. J. Hum. Genet. 52:854-865 (1993)). There are also kits that are commercially available and that provide protocols for performing FISH assays (available from e.g., Oncor, Inc., Gaithersburg, MD). Patents providing guidance on methodology include U.S. Pat. Nos. 5,225,326; 5,545,524; 6,121,489 and 6,573,043. All of these references are hereby incorporated by reference in their entirety and may be used along with similar references in the art and with the information provided in the Examples section herein to establish procedural steps convenient for a particular laboratory.

Different kinds of biological assays are called microarrays including, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes or transcripts (e.g., MMP12) by comparing gene expression in disease and normal cells or other populations. Microarrays can be fabricated using a variety of technologies, including but not limiting: printing with fine-pointed pins onto glass slides; photolithography using pre-made masks; photolithography using dynamic micromirror devices; ink-jet printing; or, electrochemistry on microelectrode arrays.

Southern and Northern blotting is used to detect specific DNA or RNA sequences, respectively. DNA or RNA extracted from a sample is fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

Nucleic acids may be amplified prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

In some embodiments, levels of MMP12 polypeptides are detected (e.g., using immunoassays or mass spectrometry).

Illustrative non-limiting examples of immunoassays include, but are not limited to: immunoprecipitation; Western blot; ELISA; immunohistochemistry; immunocytochemistry; flow cytometry; and, immuno-PCR. Polyclonal or monoclonal antibodies detectably labeled using various techniques known to those of ordinary skill in the art (e.g., colorimetric, fluorescent, chemiluminescent or radioactive) are suitable for use in the immunoassays. Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinyldiflroride or nitrocellulose, where they are probed using antibodies specific to the protein of interest. As a result, researchers can examine the amount of protein in a given sample and compare levels between several groups.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immuno-polymerase chain reaction (IPCR) utilizes nucleic acid amplification techniques to increase signal generation in antibody-based immunoassays. Because no protein equivalence of PCR exists, that is, proteins cannot be replicated in the same manner that nucleic acid is replicated during PCR, the only way to increase detection sensitivity is by signal amplification. The target proteins are bound to antibodies which are directly or indirectly conjugated to oligonucleotides. Unbound antibodies are washed away and the remaining bound antibodies have their oligonucleotides amplified. Protein detection occurs via detection of amplified oligonucleotides using standard nucleic acid detection methods, including real-time methods.

Mass spectrometry has proven to be a valuable tool for the determination of molecular structures of molecules of many kinds, including biomolecules, and is widely practiced today. Purified proteins are digested with specific proteases (e.g. trypsin) and evaluated using mass spectrometry. Many alternative methods can also be used. For instance, either matrix-assisted laser desorption/ionization (MALDI) or electrospray ionization (ESI) mass spectrometric methods can be used. Furthermore, mass spectroscopy can be coupled with the use of two-dimensional gel electrophoretic separation of cellular proteins as an alternative to comprehensive pre-purification. Mass spectrometry can also be coupled with the use of peptide fingerprint database and various searching algorithms. Differences in post-translational modification, such as phosphorylation or glycosylation, can also be probed by coupling mass spectrometry with the use of various pretreatments such as with glycosylases and phosphatases. All of these methods are to be considered as part of this application.

In some embodiments, electrospray ionization quadrupole mass spectrometry is utilized to detect MMP12 levels (See e.g., U.S. Pat. No. 8,658,396; herein incorporated by reference in its entirety).

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a blood or blood product sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., MMP12 and/or other marker expression levels), specific for the information desired for the subject.

The profile data is then prepared in a format suitable for interpretation by a treating clinician. For example, rather than providing raw expression data, the prepared format may represent a assay result (e.g., MMP12 level) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease or as a companion diagnostic to determine a treatment course of action.

Compositions for use in the screening, diagnostic, prognostic, and therapeutic methods described herein include, but are not limited to, probes, amplification oligonucleotides, and the like. In some embodiments, compositions are provided in the form of a kit. In some embodiments, kits include all components necessary, sufficient or useful for detecting the markers described herein (e.g., reagents, controls, instructions, etc.). The kits described herein find use in research, therapeutic, screening, and clinical applications.

The probe and antibody compositions of the present invention may also be provided in the form of an array.

In some embodiments, the present invention provides one or more nucleic acid probes or primers having 8 or more (e.g., 10 or more, 12 or more, 15 or more, 18 or more, etc.) nucleotides, and that specifically bind to nucleic acids encoding MMPP12.

Embodiments of the present invention provide complexes of MMP12 nucleic acids or polypeptides with nucleic acid primers or probes or antibodies. In some embodiments, a reaction mixture comprising an MMP12 polypeptide and an antibody that specifically binds to MMP12 is provided. In some embodiments, the present invention provides a multiplex (e.g., microarray) comprising reagents that binds to MMP12 and one or more additional amino acid or nucleic acids.

### EXPERIMENTAL

The following examples are provided in order to demonstrate and further illustrate certain preferred embodiments and aspects of the present invention and are not to be construed as limiting the scope thereof.

### Example 1

### Materials and Methods

### Stroke Surgeries

Stroke was induced in mice using the distal middle cerebral artery occlusion+hypoxia model. To induce stroke, animals were anesthetized by isoflurane inhalation and kept at 37° C. throughout the surgical procedure. For all experiments, mice were injected subcutaneously (s.c.) with a single dose of buprenorphine hydrochloride (0.1 mg/kg; Henry Schein) and a single dose of cefazolin antibiotic (25 mg/kg; Sigma-Aldrich) dissolved in sterile saline. Following pre-operative preparation, the skull was exposed by creating a surgical incision in the skin and temporalis muscle. The right middle cerebral artery was visually identified, and a microdrill was used to expose it. The meninges were cut, and the vessel was cauterized using a small vessel cauterizer (Bovie Medical Corporation). Surgical wounds were closed using Surgi-lock 2oc tissue adhesive (Meridian Animal Health). The mice were then transferred to a hypoxia chamber (Coy Laboratory Products) containing 9% oxygen and 91% nitrogen for 45 min. Sustained-release buprenorphine (Bup-SR, 1 mg/kg s.c.; ZooPharm) was administered 24 h after surgery as post-operative analgesia.

### RNA Sequencing and Data Analysis

Fresh brain tissue was immersed in RNAlater (Invitrogen, Cat. No. AM7020) and delivered to the University of Arizona Genetics Core. Samples were assessed for quality with an Advanced Analytics Fragment Analyzer (High Sensitivity RNA Analysis Kit, Cat. No. DNF-491/User Guide DNF-491-2014AUG13) and quantity with a Qubit RNA HS Assay Kit (Cat. No. Q32852). Given satisfactory quality (RNA integrity number >8) and quantity, samples were used for library construction with the TruSeq Stranded mRNA Library Prep Kit from Illumina (Cat. No. 20020595), as well as the KAPA Dual-Indexed Adapter Kit from Roche (Cat. No. 8278555702). Upon completion of library construction, samples were assessed for quality and average fragment size with the Advanced Analytics Fragment Analyzer (High Sensitivity NGS Analysis Kit, Cat. No. DNF-846/User Guide DNF-486-2014MAR10). Quantity was assessed with an Illumina Universal Adaptor-specific qPCR kit from KAPA Biosystems (KAPA Library Quantification Kit for Illumina NGS, Cat. No. KK4824/KAPA Library Quantification Technical Guide—August 2014). Following the final library quality control, samples were equimolar-pooled and clustered for paired-end sequencing on the Illumina NextSeq500 machine to generate 75 bp reads. The sequencing run was performed using Illumina NextSeq500 run chemistry (NextSeq500/550 High Output v2 Kit 150 cycles, Cat. No. FC-404-2002). Sequencing data is publicly available at the National Center for Biotechnology Information through Gene Expression Omnibus accession numbers: GSE173544 and GSE173715. For data analysis, the resulting sequences were demultiplexed using bcl2fastq v2.19 (Illumina) and trimmed of their indexing adaptors using Trimmomatic v0.32 (Bolger, Lohse, & Usadel, 2014). The trimmed reads were aligned to the GRCm38 reference genome using STAR v2.5.2b (Dobin et al., 2013). Gene expression was calculated using the htseq-count function of the HTSeq python tool (Anders, Pyl, & Huber, 2015). Genes were annotated using the BioMart database. Differential expression analysis of count tables was performed using DESeq2 (Love, Huber, & Anders, 2014). Gene set enrichment analysis (GSEA) was performed on all significant differentially expressed genes (false discovery rate [FDR]-adjusted p<0.05) using a database of Gene Ontology (GO) terms for biological processes. Enrichment maps were constructed from GO terms using the Enrichment Map Cytoscape application (Merico, Isserlin, Stueker, Emili, & Bader, 2010). Minimal editing, such as repositioning of nodes and removal of repetitive gene-sets, was performed to optimize the map layout. Pathway analysis was performed on differentially expressed genes using Ingenuity Pathway Analysis v01-13 (IPA).

### Multiplex Immunoassay Protocol

Total concentrations of matrix metalloproteinases (MMP) and cytokines/chemokines in flash-frozen plasma samples were measured using mouse multiplex magnetic bead kits (Milliplex Multiplex Assays, Millipore Sigma) as directed by the manufacturer. Standards, and quality controls were measured in duplicate. Plates were read using a MAGPIX instrument (Luminex), and results were analyzed using MILLIPLEX Analyst 5.1 software (Millipore Sigma).

### Results

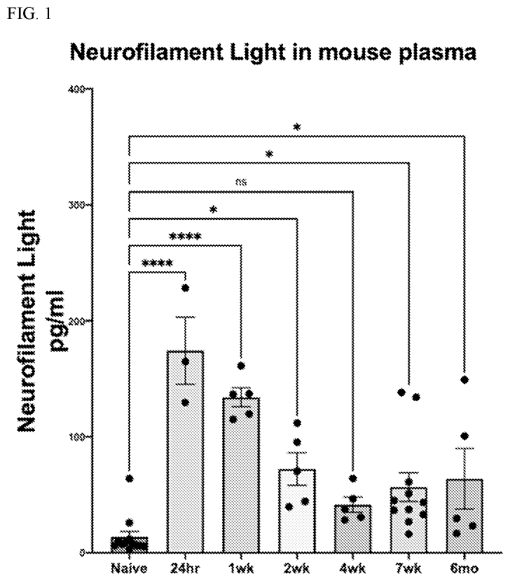

Neurofilament Light, a biomarker of neurodegeneration, is elevated in the plasma for several weeks following experimental stroke in mice (assessed by single molecule array technology: SIMOA) (FIG. 1). This demonstrates that neurodegeneration after stroke persists for weeks beyond the initial ischemic event.

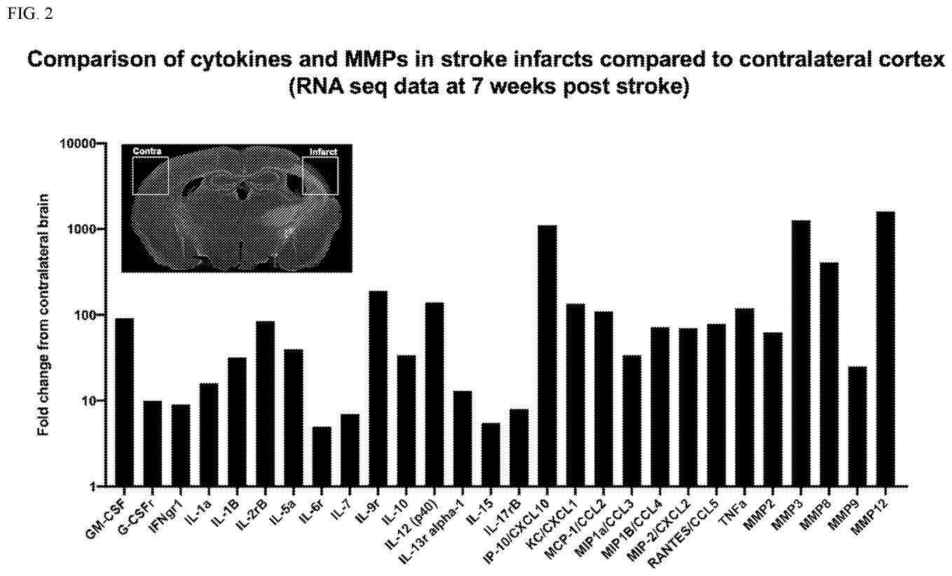

RNASeq data from the infarct (area of brain injury) at 7 weeks following stroke provides a list of analytes elevated several hundred to several thousand-fold over contralateral (uninjured control) brain tissue (www.biorxiv.org/content/10.1101/2021.05.03.442388v1) (FIG. 2). This provides a list of biomarkers that may be related to the chronic inflammatory response to stroke.

For example, MIP-2 and RANTES were increased in the infarct approximately 70-fold and 80-fold, respectively. MIP-2 is secreted by macrophages and responsible for the recruitment and activation of neutrophils during inflammation. Similarly, RANTES is chemotactic for T cells and recruits leukocytes to inflammatory sites.

Both keratinocytes-derived chemokine (KC) and Interleukin 12 subunit p40 [IL-12 (p40)] were also increased in the infarct approximately 140-fold over contralateral brain. KC (also known as CXCL1) is a chemoattractant for several immune cells to the site of injury and regulates inflammatory responses. IL-12 (p40) is a cytokine that is expressed by activated macrophages and acts on T and Natural Killer cells.

Significantly, matrix metalloproteinase-12 (MMP12) was increased approximately 1600-fold at 7 weeks post stroke in the infarct. Matrix metalloproteinases are involved in the breakdown of extracellular matrix proteins. MMP12 is also known as the macrophage metalloelastase gene and degrades substrates that include elastin, fibronectin, and Type IV Collagen.

Using the RNASeq data as a guide, a multiplex immunoassay analysis of mouse blood at 24 hours, 1 week, 2 weeks, 4 weeks, 7 weeks, and 6 months after stroke was performed to identify biomarkers of the chronic inflammatory response to stroke. Plasma samples were compared to plasma from naïve mice as a reference control.

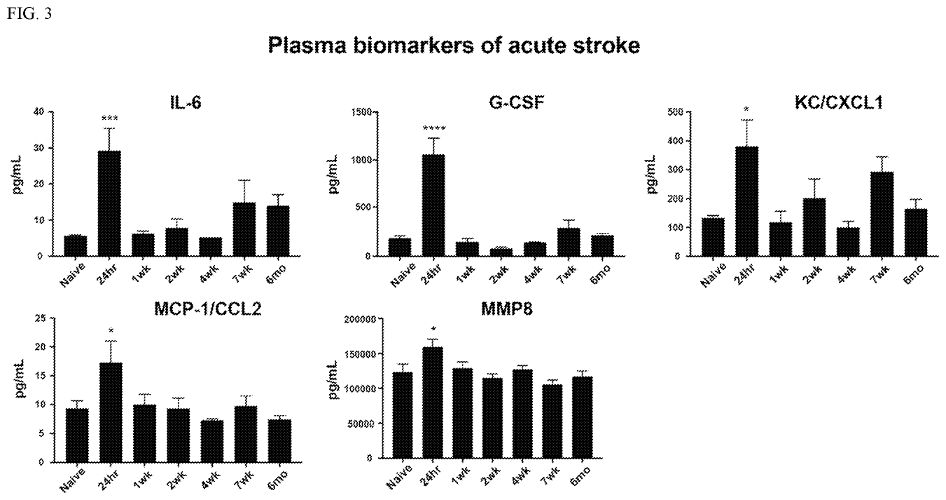

This revealed that IL-6, G-CSF, KC (CXCL1), MCP-1 (CCL2), and MMP8 are biomarkers of the acute (24 hrs) inflammatory response to stroke (FIG. 3).

The immunoassay data also revealed that MMP12 is a biomarker of the chronic inflammatory response to stroke, with elevated levels detectable in the plasma at 2 weeks, 4 weeks, 7 weeks, and 6 months after stroke.

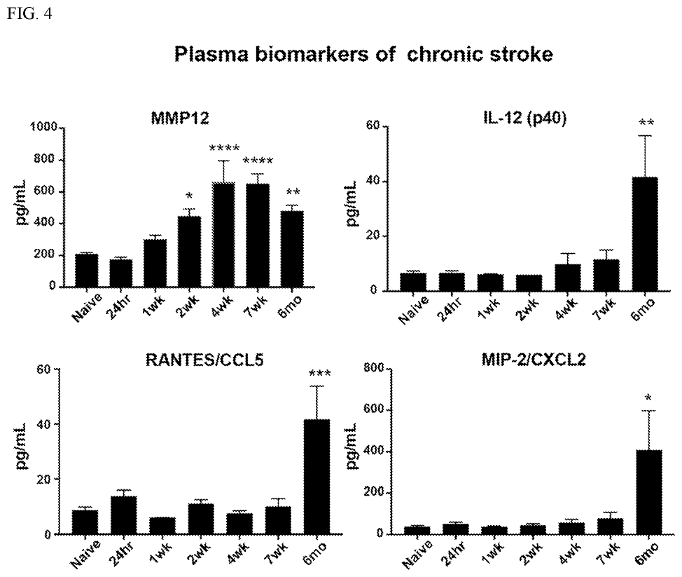

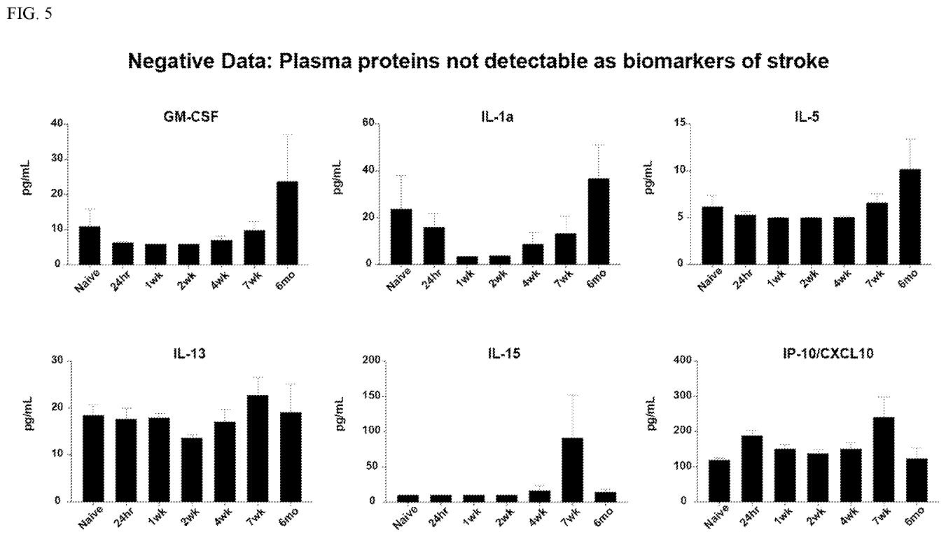

Levels of IL-12(p40), RANTES, and MIP-2 (CXCL2) were also significantly elevated 6 months following stroke, which indicates that these are plasma biomarkers of infarct resolution and end-stage fibrosis (FIG. 4).

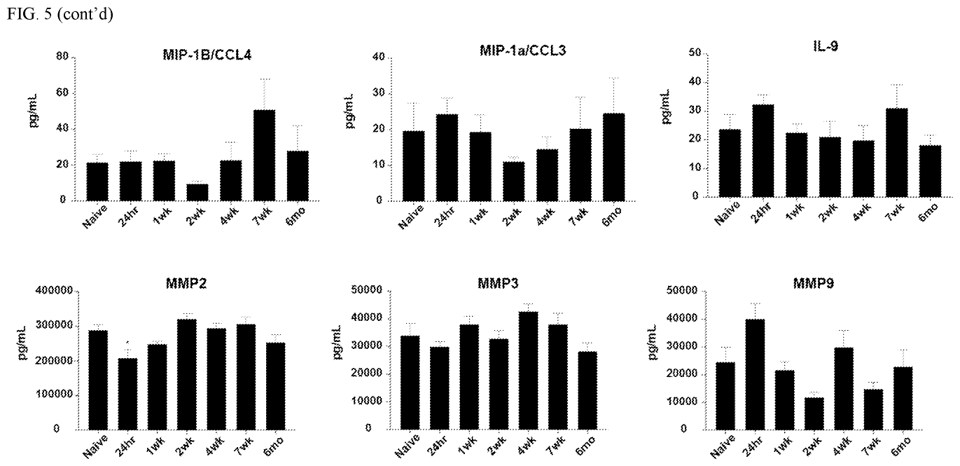

Despite their transcript levels being significantly elevated in the brain at 7 weeks following stroke, some analytes did not show significance in plasma at the different timepoints (GM-CSF, IL-1a, IL-5, IL-13, IL-15, IP-10/CXCL10, MIP-1B/CCL4, MIP-1a/CCL3, IL-9, MMP2, MMP3, and MMP9) (FIG. 5). Other analytes such as IFNg, IL-1B, IL-2, IL-7, IL-10, IL-17, and TNFa were not detectible in the plasma at any timepoint after stroke.

Together, these data support that MMP12 is a biomarker for assessing chronic inflammation after stroke, and when combined with an established plasma marker of neurodegeneration, such as Neurofilament Light, is a biomarker of chronic inflammation and neurodegeneration after stroke.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1. (canceled)

2. A method of treating chronic inflammation in a subject that has had a stroke, comprising:
a) assaying a sample from a subject that has had a stroke for the level of MMP12; and
b) administering an anti-inflammation therapy when the level of MMP12 is increased relative to the level in a subject that has not had a stroke.

3. A method of treating chronic inflammation in a subject that has had a stroke, comprising:
a) assaying a sample from a subject that has had a stroke for the level of MMP12;
b) administering an anti-inflammation therapy when the level of MMP12 is increased relative to the level in a subject that has not had a stroke; and
c) repeating said assaying step after said administering.

4. A method of monitoring a treatment in a subject that has had a stroke, comprising:
a) assaying a sample from a subject that has had a stroke for the level of MMP12;
b) administering an anti-inflammation therapy when the level of MMP12 is increased relative to the level in a subject that has not had a stroke; and
c) repeating said assaying step after said administering.

5. The method of claim 2, wherein said sample is blood, a blood product, cells, or tissue.

6. The method of claim 2, wherein said stroke is an ischemic stroke.

7. The method of claim 2, wherein said level of MMP12 is the level of MMP12 nucleic acid or polypeptide.

8. The method of claim 2, further comprising measuring the level of MMP13.

9. The method of claim 2, wherein said anti-inflammation therapy is an agent that blocks the activity of a cytokine.

10. The method of claim 9, wherein said cytokine is selected from the group consisting of IL-1α, IL-1β, and IL-1Ra, and TNF-α.

11. The method of claim 9, wherein said agent is selected from the group consisting of a small molecule, a nucleic acid, and an antibody.

12. The method of claim 2, wherein said anti-inflammation therapy is selected from the group consisting of cyclodextrin, 2-hydroxypropyl-β-cyclodextrin (HPβCD), AdipoRon, biographene quantum dots, anti-CD20 antibodies, anti-TREM2 antibodies, anti-CD36 antibodies, metformin, rapamycin, inhibitors of cyclic nucleotide phosphodiesterase (PDE), dimethyl fumarate, TLR antagonists and modulators, dimethyl itaconate, and neurotrophin receptor modulators.

13. The method of claim 2, wherein said assaying is performed at a time period of one or more of 24 hours, 1 week, 2 weeks, 4 weeks, 7 weeks, and 6 months after stroke.

14. The method of claim 13, wherein said assaying is repeated at an interval of 1 day, 1 week, one month, or one year.

15. The method of claim 2, further comprising assaying the level of a marker of neurodegeneration.

16. The method of claim 15, wherein said marker of neurodegeneration is neurofilament light (NfL).

17. The method of claim 2, wherein said anti-inflammation therapy reduces or prevents neurodegeneration in said subject.

18. (canceled)
